(19) United States
(12) Patent Application Publication
(10) Pub. No.: US 2025/0387070 A1
(43) Pub. Date: Dec. 25, 2025

(54) AI-POWERED EEG SYSTEM WITH PATHWAY HIERARCHICAL ADAPTIVE REFERENCING FOR LOCALIZED DETECTION, AUTOMATED REPORTING, AND IOMT-ENABLED ADAPTIVE NEUROMODULATION

(71) Applicants: Mohammed Abouelsoud, Mayfield Heights, OH (US); David J. Mishelevich, Playa del Rey, CA (US)

(72) Inventors: Mohammed Abouelsoud, Mayfield Heights, OH (US); David J. Mishelevich, Playa del Rey, CA (US)

(73) Assignee: U LLC, Mayfield Heights, OH (US)

(21) Appl. No.: 19/245,918

(22) Filed: Jun. 23, 2025

Related U.S. Application Data
(60) Provisional application No. 63/663,300, filed on Jun. 24, 2024.

(51) Int. Cl. A61B 5/384; A61B 5/00; G16H 15/00; G16H 50/20

(52) U.S. Cl. CPC A61B 5/384; A61B 5/0042; A61B 5/4836; A61B 5/7203; A61B 5/742; G16H 15/00; G16H 50/20; A61B 2562/046

(57) ABSTRACT

The present invention describes an artificial intelligence (AI) enabled electroencephalography (EEG) system that integrates Pathway Hierarchical Adaptive Referencing (PHAR) for localized signal detection, large language models (LLMs) for automated EEG reporting, and Internet of Medical Things (IoMT) connectivity for adaptive neuromodulation control. The system can also deliver transcranial electrical stimulation (tES) pulses and function as an electrical impedance tomography (EIT) system. PHAR employs a multi-layered multiplexer hierarchy and adaptive referencing topologies to optimize EEG signal acquisition and spatial resolution. LLM integration enables automated generation of human-readable EEG reports. IoMT connectivity allows closed-loop neuromodulation, where real-time EEG analysis guides the adjustment of stimulation parameters. The system can deliver tES pulses and perform EIT expands its functionality, allowing for targeted neuromodulation and impedance-based brain imaging. This integrated system revolutionizes EEG-based diagnostics, treatment, and research in neurology and neuroscience, offering a comprehensive and versatile tool for understanding and modulating brain function.

### CROSS-REFERENCE TO RELATED APPLICATIONS

This is a provisional patent application and does not claim priority to any other patent application.

### INCORPORATION BY REFERENCE

All publications, including patents and patent applications, mentioned in this specification are herein incorporated by reference in their entirety to the same extent as if each individual publication was specifically and individually cited to be incorporated by reference. This includes U.S. Pat. No. 11,634,281.

### FIELD OF THE INVENTION

Described herein are systems for using Pathway Hierarchical Adaptive Referencing processing of EEG signals, including Artificial-Intelligence elements for automated EEF interpretative reporting and as the basis for neuromodulation.

### BACKGROUND

### Conventional EEG Systems and Their Limitations

Conventional electroencephalography (EEG) systems have been instrumental in measuring and analyzing brain electrical activity for diagnostic and research purposes. However, these systems face several limitations. Traditional EEG electrode arrays have a relatively low spatial resolution, making it challenging to precisely localize neural sources and map brain activity with high granularity. Additionally, these systems often struggle with artifacts and noise contamination, which can obscure the underlying neural signals of interest.

Furthermore, conventional EEG systems lack adaptability and real-time optimization capabilities. The electrode configurations and referencing schemes are typically fixed, unable to dynamically adjust to changing signal characteristics or individual variations in brain anatomy and neurophysiology. This lack of adaptability can result in suboptimal signal quality and limited insight into the complex spatiotemporal dynamics of brain activity.

### Advancements in AI, LLMs, and IoMT Technologies

Recent years have witnessed significant advancements in various technologies that hold the potential to address the limitations of conventional EEG systems. Artificial intelligence (AI) and machine learning techniques have made considerable strides, enabling more sophisticated analysis and interpretation of complex neural data. Large language models (LLMs) have emerged as powerful tools for natural language processing, opening up new avenues for automated EEG reporting and enhancing interpretability for clinicians and researchers.

Concurrently, the Internet of Medical Things (IoMT) has gained traction, facilitating the integration of diverse medical devices, wearables, and remote monitoring systems into interconnected healthcare ecosystems. This interconnectivity holds promise for real-time data sharing, enabling closed-loop feedback systems and adaptive treatment paradigms.

### Unmet Needs in EEG-Based Diagnostics, Treatment, and Research

Despite the advancements in AI, LLMs, and IoMT technologies, several unmet needs remain in the realm of EEG-based diagnostics, treatment, and research:
High-resolution EEG mapping: There is a pressing need for EEG systems capable of providing high spatial resolution and precise localization of neural sources, enabling more accurate diagnosis and targeted interventions.
Artifact removal and noise suppression: Robust techniques are required to effectively remove artifacts and suppress noise from EEG signals, ensuring a clear representation of the underlying neural activity.
Adaptive and personalized EEG analysis: Existing EEG systems lack the ability to adapt to individual variations in brain anatomy and neurophysiology, limiting the accuracy and personalization of diagnosis and treatment.
Real-time closed-loop feedback: There is a need for EEG or EIT systems that can operate in a cl[0003] osed-loop manner, continuously monitoring brain activity and providing real-time feedback to guide adaptive interventions or neuromodulation therapies.
Integration with AI and LLMs: Leveraging the power of AI and LLMs can enhance the interpretation and reporting of EEG data, making it more accessible and actionable for clinicians and researchers.
Seamless integration with IoMT ecosystems: Integrating EEG systems with IoMT networks can facilitate remote monitoring, data sharing, and the development of personalized treatment protocols based on a comprehensive understanding of an individual's neurological and physiological data.

Addressing these unmet needs holds the potential to revolutionize EEG-based diagnostics, treatment, and research, enabling more precise, personalized, and effective interventions for a wide range of neurological conditions.

### SUMMARY OF INVENTION

The present invention provides an AI-powered EEG system that achieves unprecedented spatial precision and adaptability in localizing EEG signal detection within an ultra-dense electrode array. The core innovations are: 1) the Pathway Hierarchical Adaptive Referencing circuit that dynamically optimizes electrode clustering for maximal signal quality, 2) the integration of AI machine learning techniques for artifact removal, source localization, and EEG classification, 3) the integration with large language models (LLMs) for automated generation of human-readable EEG reports, and 4) the integration with Internet of Medical Things (IoMT) networks for closed-loop adaptive neuromodulation control.

The high-density electrode array contains 128-1024 or more EEG sensors to enable high-resolution spatial sampling across the scalp. The Pathway Hierarchical Adaptive Referencing circuit employs a multi-layered multiplexer hierarchy to flexibly configure these sensors into dynamically-adjusted clusters based on real-time EEG characteristics and clinical information, such as the location of epilepsy foci. By incorporating patient-specific clinical data, the system can predict and prioritize referencing topologies that are most likely to capture relevant neural activity. For example, if a patient has a known epileptogenic zone in the temporal lobe, the referencing scheme can be biased towards electrode configurations that provide high spatial resolution and signal-to-noise ratio in that region. Parallel processing units use signal quality metrics and clinical priors to evaluate a vast number of possible referencing topologies and converge on the optimal configuration at any given moment. This allows the system to adaptively focus on and isolate true neural sources while suppressing artifacts and noise, guided by both real-time EEG dynamics and individual patient characteristics. The integration of clinical information enhances the specificity and sensitivity of the PHAR system, enabling personalized and clinically relevant EEG acquisition and analysis.

The AI component leverages state-of-the-art machine learning models, such as, but not limited to, convolutional neural networks (CNNs), recurrent neural networks (RNNs), generative adversarial networks (GANs), and graph neural networks (GNNs), which are trained on large EEG datasets. These models perform critical functions including noise reduction, eye blink and muscle artifact removal, and EEG source localization to precisely map surface potentials to originating neural structures.

CNNs excel at learning spatial hierarchies and extracting relevant features from EEG topographies, while RNNs capture temporal dependencies and sequential patterns in EEG time series. GANs enable the generation of realistic EEG data for data augmentation and simulation purposes, enhancing the robustness and generalizability of the AI models.

Furthermore, the incorporation of GNNs allows the AI component to effectively model and analyze the complex graph-structured relationships between EEG channels, cortical regions, and functional brain networks. GNNs can learn the intricate spatial and temporal dependencies in EEG data, taking into account the topological structure of the brain and the dynamic interactions between different neural populations.

By leveraging GNNs, the AI component can capture the rich interconnectivity of brain regions and identify key patterns and biomarkers that may be overlooked by traditional machine learning approaches. This enables more accurate and comprehensive EEG analysis, facilitating the detection of subtle neurological abnormalities, the identification of functional connectivity patterns, and the prediction of clinical outcomes.

The AI system also classifies EEG spatiotemporal patterns to decode high-level neural dynamics and brain states related to cognition, perception, and various neurological conditions. By integrating information from multiple GNN layers, the AI component can hierarchically abstract EEG features and build robust representations of brain activity across different spatial and temporal scales.

The inclusion of GNNs in the AI component expands the coverage and versatility of the EEG analysis pipeline, allowing for a more comprehensive understanding of brain function and dysfunction. It enables the exploration of complex brain network dynamics, the identification of disease-specific biomarkers, and the development of personalized diagnostic and therapeutic strategies based on individual brain connectivity profiles.

The integration with large language models (LLMs) enables automated generation of human-readable EEG reports, enhancing the interpretability and clinical utility of the EEG findings. The LLMs are fine-tuned on a corpus of expert-annotated EEG reports and EEG data, allowing them to generate coherent and informative reports describing key patterns, interpretations, and potential implications of the EEG analysis.

The inclusion of an electrical impedance tomography (EIT) component allows for impedance-based brain imaging to provide feedback for adjustment of neuromodulation parameters.

The IoMT integration allows for closed-loop adaptive neuromodulation, where real-time analysis of EEG data guides the adjustment of stimulation parameters in connected devices, such as transcranial electrical stimulation (tES) systems or implanted neuromodulation devices. This closed-loop feedback enables personalized and responsive modulation of neural activity, optimizing therapeutic outcomes and treatment efficacy.

Furthermore, the AI-powered EEG system can connect with various other IoMT devices to provide a comprehensive and integrated approach to patient care and treatment. For instance, the system can interface with respiration control devices, such as ventilators or continuous positive airway pressure (CPAP) machines, to monitor and adjust respiratory parameters based on EEG-derived indicators of brain function and sleep quality.

In the context of neurorehabilitation, the system can connect with MRI scanners and utilize real-time EEG data to guide the selection of MRI pulse parameters, ensuring optimal image acquisition and minimizing artifacts. This integration allows for simultaneous EEG-fMRI recordings, providing valuable insights into the spatiotemporal dynamics of brain activity and connectivity.

The AI-powered EEG system can also interface with paired associative stimulation (PAS) neuromodulation devices, which combine peripheral nerve stimulation with transcranial magnetic stimulation (TMS) to induce neuroplasticity. By integrating real-time EEG data, the PAS parameters can be dynamically adjusted to maximize the effectiveness of the neuromodulation protocol and promote targeted neural reorganization.

Moreover, the system can seamlessly connect with electronic health record (EHR) systems, enabling the automatic transfer of EEG findings, automated reports, and treatment recommendations to the patient's medical history. This integration streamlines clinical workflows, facilitates data-driven decision making, and enhances care coordination among healthcare providers.

In the realm of cardiology, the AI-powered EEG system can establish connections with implantable pacemakers and other cardiac devices to monitor and modulate brain-heart interactions. By analyzing EEG data in conjunction with cardiac parameters, the system can identify abnormal patterns and trigger appropriate interventions to maintain optimal cardiovascular function.

The extensive IoMT connectivity of the AI-powered EEG system enables a holistic and multidisciplinary approach to patient management, integrating neurological, respiratory, rehabilitative, and cardiac care. By leveraging real-time data from multiple IoMT devices and adapting stimulation parameters accordingly, the system offers unprecedented opportunities for personalized, adaptive, and comprehensive neuromodulation therapies.

The software architecture efficiently integrates the Pathway Hierarchical Adaptive Referencing, AI components, LLM-based reporting, and IoMT connectivity for real-time operation. A powerful CPU and GPU enable rapid processing of the multi-channel EEG data stream. An interactive user interface supports 3D visualization of high-resolution EEG activity maps, while an API allows flexible integration with various external systems, including electronic health records (EHRs) and IoMT devices.

In summary, the AI-powered EEG system combined with one or more of Pathway Hierarchical Adaptive Referencing (PHAR), automated EEG reporting, EIT monitoring, and IoMT-enabled adaptive neuromodulation sets a new standard for high-density EEG acquisition, analysis, and closed-loop intervention. By optimizing the entire data pipeline from dynamic electrode configurations to machine learning-driven artifact removal, source localization, spatiotemporal decoding, and personalized stimulation control, it achieves unparalleled resolution in mapping and modulating the neural correlates of brain function. This technology has immense potential to advance the frontiers of neurology, cognitive neuroscience, brain-computer interfacing, and personalized neurotherapeutics.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates a block diagram of Pathway Hierarchical Adaptive Referencing Processing steps for the processing of EEG signals.

FIG. 2 shows breakdowns into additional elements for selected elements of FIG. 1.

FIG. 3 illustrates a hardware diagram of a configuration for the support of the Pathway Hierarchical Adaptive Referencing processing of EEG signals.

FIG. 4 shows locations of EEG electrodes on the surface of the scalp in a conventional 10-20 configuration to which additional electrode positions have been added to provide for higher resolution.

FIG. 5 shows a cross section of pogo pins at various lengths of extension to accommodate a curved surface.

FIG. 6 shows a portion of a high-density EEG-recording electrode array each with a different set of electrodes activated.

FIG. 7 illustrates the automated generation of EEG-interpretation reports.

FIG. 8. shows a table of use cases in various domains.

### DETAILED DESCRIPTION OF THE INVENTION

2. AI-Powered EEG System with Pathway Hierarchical Adaptive Referencing (PHAR)

### 2.1. System Overview and Architecture

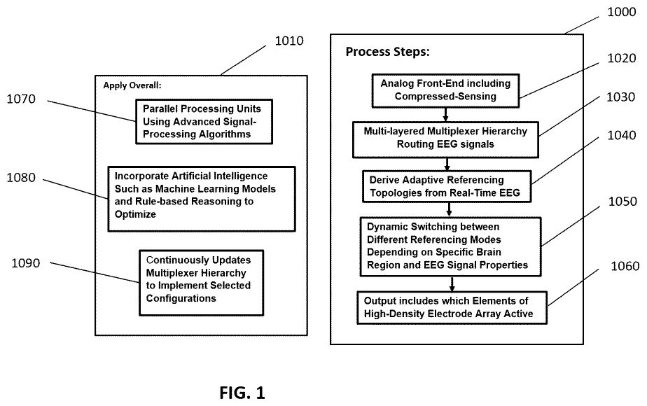

The AI-powered EEG system with Pathway Hierarchical Adaptive Referencing (PHAR) is designed to optimize the acquisition and processing of high-density EEG signals. The system dynamically adapts the referencing scheme and electrode configurations in real-time to maximize signal quality, minimize noise and artifacts, and enhance the spatial resolution of EEG source localization. FIG. 1 is divided into two parts. Process Steps box 1000 on the right diagrams the steps Analog Front-End including Compressed-Sensing 1020, providing input to Multi-layered Multiplexer Hierarchy Routing EEG signals 1030, Derive Adaptive Referencing Topologies from Real-Time EEG 1040, Dynamic Switching between Different Referencing Modes Depending on Specific Brain Region and EEG Signal Properties 1050, and Output includes which Elements of High-Density Electrode Array Active 1060. On the left, box 1010 contains elements that are applied throughout the processing in Process Steps box 1000 including Parallel Processing Units Using Advanced Signal-Processing Algorithms 1070, Incorporate Artificial Intelligence Such as Machine Learning Models and Rule-based Reasoning to Optimize 1080, and Continuously Updates Multiplexer Hierarchy to Implement Selected Configurations 1090.

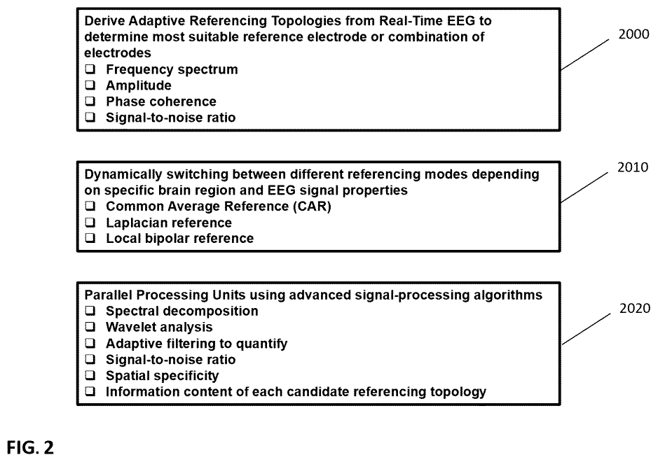

FIG. 2 shows a breakdown of some of the elements in Process Steps box 1000. Box 2000 lists the bases of Derived Referencing Topologies related to box 1040 in FIG. 1. Box 2010 illustrates elements of Switching between different referencing modes related to box 1050 in FIG. 1. Box 2020 lists signal-processing algorithms applied by Parallel Processing Units related to box 1070 in FIG. 1.

### Implementation of System

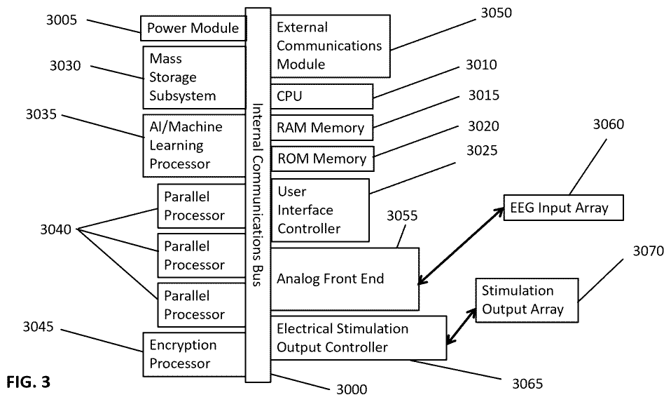

FIG. 3 illustrates a hardware embodiment to support PHAR processing. This embodiment is one of many possible configurations. Internal communications are handled on Internal Communications Bus 3000, although, alternatively, selected direct connections between or among components are possible. Power Module 3005 supports the entire system. General Purpose CPU 3010 orchestrates the interactions in addition to providing processing with storage provided by RAM 3015, ROM 3025, and Mass Storage Subsystem 3030. Special purposes processors such as Parallel Processors 3040 (or other Artificial Intelligence processor, not shown) and Encryption Processor 3045 can be included. User interface interactions are provided via User Interface Controller 3025. Input/Output is provided by External Communications Module 3050, Analog Front End 3055 with input from EEG Input Array 3060 and Electrical Stimulation Output Controller 3065 with neuromodulation output via Stimulation Output Array 3070 noting that electrodes for both EEG input and stimulation output can be shared, typically by use of a multiplexer (not shown).

### 2.2. Ultra-Dense Electrode Array and Signal Acquisition

The PHAR system is designed to operate with ultra-dense electrode arrays containing 128-1024 or more individual sensors, with a typical inter-electrode spacing of 5 to 20 mm or less. The system can accommodate various electrode configurations, including conventional 10-20 systems, high-density geodesic arrays, and custom montages tailored for specific applications. The analog front-end of the PHAR system features low-noise amplifiers with a wide dynamic range (>120 dB) and high input impedance (>1 GΩ) to ensure accurate capture of EEG signals across a broad range of amplitudes and frequencies. Furthermore, to prevent cross-talk between the ab-electrode, a compress sensing randomization scheme is deployed when sampling across the sub-electrodes to prevent any two adjacent sub-electrodes being sampled at the same time. The system supports a sampling rate of up to 20 kHz per channel, enabling high temporal resolution and capture of fast neural dynamics.

### 2.3. Pathway Hierarchical Adaptive Referencing (PHAR)

### 2.3.1. Multi-Layered Multiplexer Hierarchy

At the heart of the PHAR system lies a sophisticated multi-layered multiplexer hierarchy that serves as the backbone for flexible electrode configuration and referencing. This hierarchy consists of 4-12 or more layers, each composed of high-speed analog multiplexers capable of switching between multiple input signals at rates up to 100 MHz. The hierarchy is designed to accommodate the ultra-dense electrode array (e.g., a dense pogo pin array), enabling seamless routing of EEG signals from the 128-1024 or more individual sensors to the downstream amplification and digitization stages. The multiplexer hierarchy is organized in a tree-like structure, with progressive aggregation of signals from the lowest layer (individual electrodes) to the highest layer (final output channels). Each multiplexer node in the hierarchy can dynamically select and switch between its input signals based on control signals from the higher-level control logic. This architecture allows for flexible, real-time reconfiguration of electrode groupings and referencing topologies without the need for physical rewiring.

### 2.3.2. Adaptive Referencing Topologies

The PHAR system employs advanced algorithms to continuously evaluate and adapt the optimal referencing topology for each localized brain region. This involves analyzing the real-time EEG signal characteristics, FIG. 2 2000, such as frequency spectrum, amplitude, phase coherence, and signal-to-noise ratio, to determine the most suitable reference electrode or combination of electrodes for each target electrode cluster. The system dynamically switches between different referencing modes, FIG. 2 2010, such as common average reference (CAR), Laplacian reference, and local bipolar reference, and inifinite referencing, depending on the specific brain region and EEG signal properties.

### 2.3.3. Parallel Processing Units

T]o efficiently explore the vast space of possible referencing topologies and electrode configurations, the PHAR system incorporates parallel processing units, FIG. 3 3040, dedicated to evaluating and optimizing the signal quality metrics. These units operate concurrently across multiple channels and hierarchical layers, enabling rapid assessment and comparison of different referencing schemes. The parallel processing units employ advanced signal processing algorithms, FIG. 2 2020, such as, but not limited to, spectral decomposition, wavelet analysis, and adaptive filtering, to quantify the signal-to-noise ratio, spatial specificity, and information content of each candidate referencing topology. They also incorporate machine learning models trained on large EEG datasets to predict the optimal referencing scheme based on learned patterns and heuristics.

### 2.3.4. Control Logic and Adaptive Configuration

The PHAR system features sophisticated control logic that orchestrates the dynamic adaptation of referencing topologies and electrode configurations based on the real-time analysis of EEG signal characteristics. The control logic receives input from the parallel processing units regarding the optimal referencing schemes and continuously updates the multiplexer hierarchy to implement the selected configurations. Selected configurations may include which one or more elements of a high-density electrode array are active. The control logic operates at multiple temporal scales, ranging from millisecond-level switching for fast artifact rejection to second-level adaptations for longer-term changes in brain state or signal quality. It employs hierarchical state machines and rule-based decision systems to determine the appropriate referencing mode and electrode groupings for each brain region and time point. Furthermore, the control logic incorporates adaptive learning algorithms that continuously refine the referencing strategies based on accumulated EEG data and performance metrics. This allows the system to learn and adapt to individual brain anatomy, neurodynamics, and signal characteristics over time, optimizing the referencing scheme for each specific subject and recording condition.

### 2.4. AI Component for EEG Analysis

### 2.4.1. Machine Learning Models for Noise Reduction, Artifact Removal, and Feature Extraction

The PHAR system seamlessly integrates with the AI component of the EEG pipeline, leveraging advanced machine-learning models for noise reduction, artifact removal, source localization, and pattern classification. The dynamically optimized referencing scheme enhances the input signal quality for these AI models, enabling more accurate and reliable EEG analysis. The AI models, such as convolutional neural networks and recurrent neural networks, are trained on extensive EEG datasets to learn robust representations of neural activity patterns and to distinguish true neural sources from noise and artifacts.

### 2.4.2. Deep-Learning Models for EEG Source Localization and Pattern Classification

The PHAR system provides these models with high-quality, spatially-localized EEG signals, facilitating precise source localization and decoding of neural dynamics. Moreover, the AI component can provide feedback to the PHAR control logic, informing the selection of optimal referencing schemes based on higher-level features and classifications derived from the EEG data. This creates a bidirectional flow of information, with the PHAR system optimizing the input signals for AI analysis, and the AI models guiding the adaptive referencing to further enhance signal quality and interpretability.

### 2.5. System Performance and Technical Specifications

The PHAR system's adaptive referencing scheme can dynamically switch between different configurations at rates up to 1 kHz, allowing for rapid adaptation to changing signal characteristics and brain states. The parallel processing units can evaluate and compare hundreds of potential referencing topologies within milliseconds, ensuring optimal signal quality and spatial specificity. The system's overall latency, from EEG signal acquisition to real-time display and output, is typically less than 10 ms, enabling near-instantaneous feedback and closed-loop applications. The PHAR architecture is highly scalable, with the capability to expand to even higher electrode densities and channel counts as sensing technologies advance.

### 2.6. Flexible Pogo Pin Electrode Array

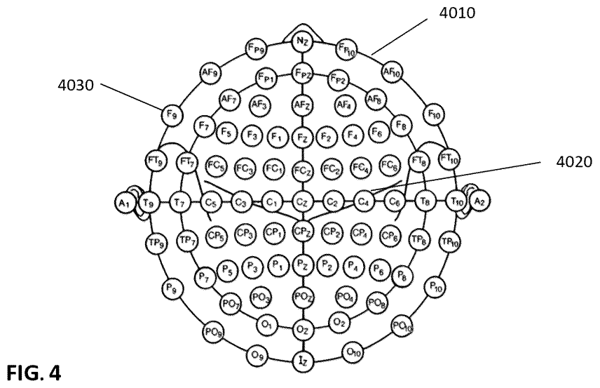

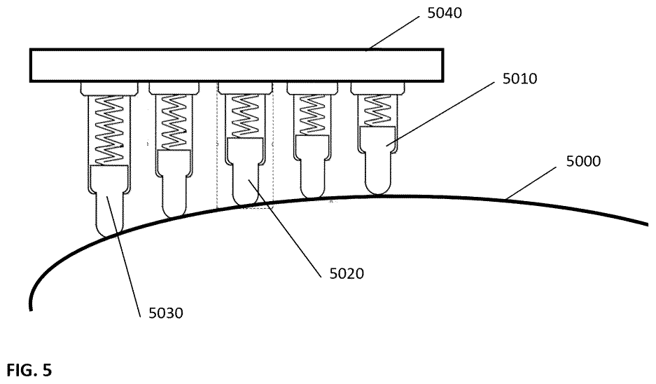

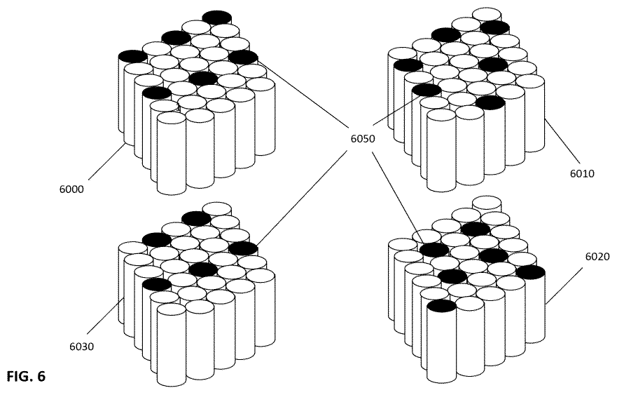

FIG. 4 shows a layout of EEG electrode positions 4010 over the skull. The base configuration is a typical 10-20 placement with an example C4 position 4020. For higher resolution, there are added elements such as the electrode F9 at position 4030. At a given location, the AI-powered EEG system incorporates a novel flexible pogo pin electrode array that enables adaptive spatial sampling and high-density EEG acquisition. The pogo pin array consists of a large number of individually addressable sub-electrodes (up to 1024 or more) arranged in a grid pattern. Each sub-electrode is a spring-loaded pogo pin that can be independently actuated and configured to operate as a singular electrode or as part of a dynamically defined cluster of sub-electrodes. The overall configuration is shown in FIG. 5 showing a cross section of a set of pogo pins distributed over a curved surface of the skull 5000. Pogo pins 5010, 5020, and 5030 illustrate various degrees of extension with all of them butted up against the flat surface 5040 which mates with the electrode-array holder. FIG. 6 shows high-density, multi-electrode arrays 6000, 6010, 6020, and 6030 in which different sets of individual electrodes 6050 (indicated by their top surfaces being filled in with black) are sequentially activated based on PHAR instructions to provide precision EEG recordings. Such electrode arrays can also be used in the same manner for neuromodulation. The overall pogo-pin array can act as a singular electrode or break-off into multiple electrodes.

The flexibility of the pogo pin array allows for seamless adaptation to different head shapes and sizes, ensuring optimal scalp contact and minimizing signal attenuation due to poor electrode-skin coupling. The individual sub-electrodes can be dynamically grouped into larger virtual electrodes of varying sizes and shapes, enabling multi-scale spatial sampling and the ability to target specific brain regions with high precision.

To prevent cross-talk between adjacent sub-electrodes and to minimize the effects of volume conduction, the system employs a compressed sensing randomization scheme when sampling across the sub-electrodes. This scheme ensures that no two adjacent sub-electrodes are sampled simultaneously, effectively reducing the spatial correlation of the acquired EEG signals. The randomization pattern is dynamically generated based on the desired spatial resolution and the targeted brain regions, optimizing the information content of the sampled data.

The pogo-pin electrode array supports ultra-high sampling rates, with the ability to acquire EEG signals at 20,000 Hz or higher. This high temporal resolution enables the capture of fast neuronal dynamics and high-frequency oscillations (HFOs) that are critical for understanding the underlying neural mechanisms of brain function and dysfunction. Recent studies have demonstrated the presence of HFOs above 500 Hz and up to 800 Hz in association with epileptic seizures. By employing high sampling rates, the AI-powered EEG system can detect and analyze these ultra-fast oscillations, providing valuable insights into the spatiotemporal dynamics of epileptogenic networks.

The combination of the flexible pogo pin electrode array, adaptive spatial sampling, compressed sensing randomization, and ultra-high sampling rates enables the AI-powered EEG system to acquire high-density, high-quality EEG data with unprecedented spatial and temporal resolution. This advanced electrode technology facilitates the precise localization of neural sources, the identification of fine-grained functional connectivity patterns, and the exploration of novel neurophysiological biomarkers for various neurological and psychiatric disorders.

3. Integration with Large Language Models for Automated EEG Reporting

### 3.1. EEG Data Preprocessing and Feature Encoding

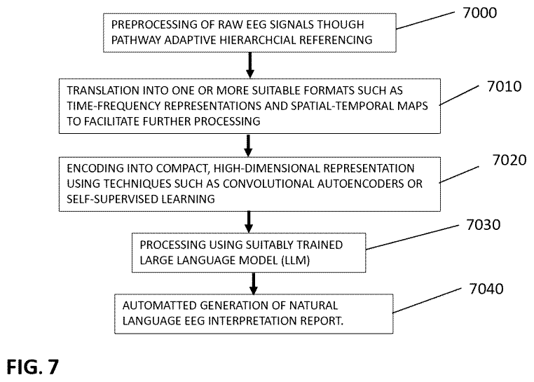

An overview of the automation of the EEG reporting system is shown in FIG. 7. The raw EEG signals acquired through the PHAR system undergo extensive preprocessing, including noise reduction, artifact removal, and feature extraction. The EEG data is preprocessed in 7000 is then transformed into a suitable format in 7010, such as time-frequency representations or spatial-temporal maps, which serve as input to the LLM. The preprocessed EEG data is encoded in 7020 into a compact, high-dimensional representation using techniques such as, but not limited to, convolutional autoencoders or self-supervised learning. This encoding captures the salient features and patterns in the EEG signals while reducing dimensionality and redundancy.

### 3.2. LLM Fine-Tuning on EEG Report Corpus

A pre-trained LLM, such as, but not limited to, GPT-3, Claude-AI, and/or BERT, is fine-tuned on a large corpus of annotated EEG reports and corresponding EEG data and applied in 7030. During fine-tuning, the LLM learns to generate coherent and informative EEG reports based on the input EEG features, leveraging its pre-existing knowledge of language structure and domain-specific terminology.

### 3.3. Automated Report Generation and Interactive Refinement

The fine-tuned LLM takes the encoded EEG features as input and in 7040 generates a natural language report describing the key findings, patterns, and interpretations of the EEG data. The report covers aspects such as dominant frequencies, spatial distribution of activity, temporal dynamics, and potential clinical implications. The generated EEG report is presented to the user through an interactive interface, allowing for further refinement and customization. The user can provide feedback, ask for clarifications, or request additional details, which the LLM incorporates to iteratively improve the report's accuracy and relevance.

3.4. Integration with Electronic Health Records (EHRs)

The final EEG report can be automatically integrated into the patient's EHR, along with the raw EEG data and visualizations. This seamless integration enables easy access to the EEG findings for clinicians and facilitates longitudinal tracking of brain function over time.

### 3.5. Benefits of Automated EEG Reporting

The benefits of integrating the AI-powered EEG system with LLMs for automated reporting are manifold. It streamlines the interpretation process, reduces the workload on EEG specialists, and ensures consistency and standardization across reports. Moreover, the LLM's ability to generate natural language explanations enhances the interpretability of EEG findings for non-specialist clinicians and patients, promoting better communication and shared decision-making.

4. Integration with IoMT Networks for Adaptive Neuromodulation

### 4.1. IoMT Connectivity and Device Discovery

The EEG system is equipped with wireless communication modules, such as Wi-Fi, Bluetooth, or cellular, which enable secure and reliable connectivity to the IoMT network. The system uses standardized communication protocols, such as MQTT or REST APIs, to exchange data and commands with other IoMT devices.

The AI-powered EEG system's IoMT (Internet of Medical Things) connectivity enables seamless integration and control of a wide range of life monitoring, life-saving, neuromodulation, and neuromonitoring devices. By analyzing real-time EEG data alongside vital signs and other physiological parameters, the system can provide intelligent control and optimization of these devices, ensuring personalized and adaptive treatment delivery. Here are some key capabilities of the system:

1. Integration with Respiratory Ventilators, CPAP Devices, Cardiac Pacemakers, Insulin Pumps, ICP Monitoring Devices, and ECMO Systems:

The system can analyze EEG patterns associated with respiratory function, cardiac autonomic function, glucose regulation, and intracranial pressure changes. It can then adjust device parameters, such as ventilator settings, CPAP pressure, pacemaker pacing, insulin delivery, and ICP management strategies, to optimize patient safety and clinical outcomes.

### 2. Control of Neuromodulation Devices:

The system can connect with devices like Transcranial Electrical Stimulation (tES), Deep Brain Stimulation (DBS) implants, Vagus Nerve Stimulation (VNS), and Responsive Neurostimulation (RNS) systems. It can dynamically adjust stimulation parameters based on the individual's EEG patterns and treatment response, implementing closed-loop control and personalized therapy delivery.

3. Integration with Wearable Neuromonitoring Devices and Electronic Health Records (EHRs):

The system can collect data from wearable devices like smartwatches and EEG headsets, as well as access clinical information from EHRs. It can then integrate this data with EEG analysis to provide a comprehensive assessment of the individual's neurological health and treatment response, enabling personalized recommendations and care coordination.

### 4. Clinical Decision Support and Automated Reporting:

The system can integrate with clinical decision support systems to provide evidence-based recommendations for diagnosis, treatment selection, and patient management based on the individual's EEG findings and clinical profile. It can also generate automated reports and alerts to facilitate timely communication between healthcare providers.

By leveraging the IoMT connectivity and interoperability, the AI-powered EEG system enables a holistic and personalized approach to critical care management, neuromodulation therapy, and patient monitoring. It can dynamically control and optimize various device parameters across multiple systems, adapting to the individual's unique neurological characteristics and treatment needs. This integrated approach enhances the precision, efficacy, and safety of medical interventions, ultimately improving patient outcomes and quality of life.

### 4.2. Real-Time Data Exchange and Adaptive Stimulation Control

The EEG system continuously streams real-time EEG data, including preprocessed signals, extracted features, and AI-generated insights, to the connected IoMT devices. This data exchange enables the IoMT devices to adapt their stimulation parameters based on the current brain state and neurodynamics.

The AI component of the EEG system analyzes the incoming EEG data and generates optimal stimulation parameters for the connected IoMT devices. These parameters, which are dynamically adjusted in real-time to maintain the desired brain state or modulate specific neural pathways, include stimulation frequency to target specific oscillatory patterns or frequency bands; intensity, such as current amplitude or voltage, to optimize the balance between therapeutic efficacy and patient comfort; temporal patterns, including pulse width, duty cycle, and inter-stimulus intervals, to synchronize with the endogenous neural activity patterns or induce specific neuroplastic changes; spatial targeting to specific brain regions or networks based on real-time EEG source localization results; waveform selection and adjustment based on EEG signal characteristics and desired neuromodulatory effects; closed-loop feedback for continuous optimization of the stimulation protocol in response to the individual's dynamic brain states and treatment progress; multi-modal integration with other physiological signals for a more comprehensive assessment and tailoring of stimulation parameters; adaptive thresholding based on EEG signal quality and artifact detection; personalized stimulation protocols optimized for each individual using machine learning algorithms; and safety constraints to ensure the tolerability of the neuromodulation and prevent potential adverse effects or discomfort. In addition to real-time data analysis, the EEG system incorporates in-silico models, such as Kuramoto models, for EEG forecasting. Kuramoto models are mathematical representations of coupled oscillators that can simulate the synchronization patterns observed in neural networks. By integrating these models, the EEG system can predict future EEG activity based on the current brain state and stimulation parameters. This predictive capability enables proactive adjustment of stimulation parameters, allowing the system to anticipate and prevent undesired brain states or to optimize the timing of neuromodulation interventions.

### 4.3. Closed-Loop Feedback and Personalized Neuromodulation

The IoMT devices provide feedback to the EEG system regarding the applied stimulation parameters and their effects on brain activity. The EEG system incorporates this feedback into its ongoing analysis, enabling adaptive fine-tuning of stimulation parameters for personalized, responsive neuromodulation.

### 4.4. Remote Monitoring and Control Capabilities

The EEG system's integration with IoMT networks enables remote monitoring and control capabilities. Clinicians and researchers can securely access the EEG data and stimulation parameters from remote locations, allowing for tele-neurology and remote treatment management. This feature is particularly valuable for patients in underserved areas or for those requiring continuous monitoring.

### 4.5. Data Security and Privacy Considerations

The integration of the EEG system with IoMT networks prioritizes data security and patient privacy. All data transmissions are encrypted using industry-standard protocols, such as TLS/SSL, and access to sensitive information is strictly controlled through role-based authentication and authorization mechanisms.

### 4.6. Applications in Adaptive Neuromodulation Therapies

The integration of the AI-powered EEG system with IoMT networks unlocks new possibilities for personalized, adaptive neuromodulation therapies. By leveraging real-time EEG analysis to guide the adjustment of stimulation parameters in connected devices, this integrated approach can optimize treatment efficacy, minimize side effects, and promote targeted modulation of neural circuits. Moreover, the ability to monitor and control brain activity remotely through IoMT networks enhances accessibility, convenience, and continuity of care for patients with neurological conditions.

### 5. Applications and Use Cases

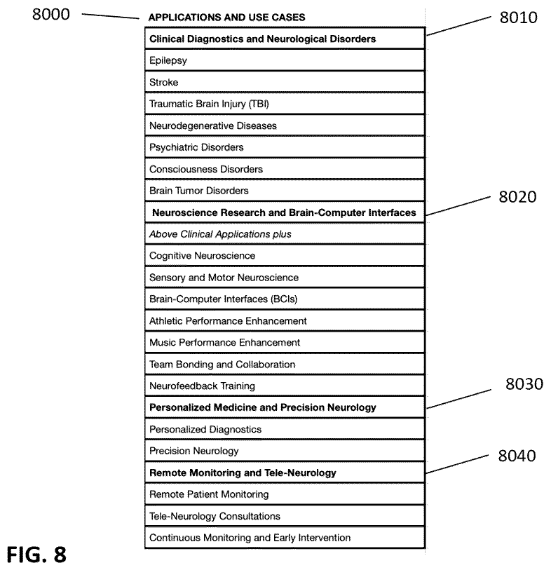

A selection of applications and use cases is listed in the table 8000 of FIG. 8.

### 5.1. Clinical Diagnostics and Neurological Disorders

The AI-powered EEG system with Pathway Hierarchical Adaptive Referencing (PHAR) holds significant potential for clinical diagnostics and the management of various neurological disorders as noted in section 8010. Its ability to provide high-resolution mapping of brain activity, coupled with advanced artifact removal and source localization capabilities, can aid in the accurate diagnosis and characterization of conditions such as epilepsy, stroke, traumatic brain injury, and neurodegenerative diseases.

The AI-powered EEG system with PHAR has a wide range of applications in diagnosing, monitoring, and guiding treatment, including surgical planning, for various neurological disorders, including:

### 1. Epilepsy:

The PHAR system can precisely localize epileptogenic foci and map the spatiotemporal dynamics of seizure propagation, facilitating surgical planning and continuous monitoring of epileptic activity.

It can guide the placement of intracranial electrodes and the extent of surgical resection, improving the accuracy and efficacy of epilepsy surgery.

The system can also monitor the EEG activity during awake craniotomy procedures, providing real-time feedback to the neurosurgeon about the functional impact of surgical manipulations.

### 2. Stroke:

By accurately identifying the affected brain regions and monitoring the evolution of neural activity patterns, the system can assist in stroke diagnosis, prognosis, and tracking of recovery processes.

It can guide the selection of appropriate neurorehabilitation strategies based on the individual's EEG profiles and monitor the effectiveness of interventions over time.

The PHAR system can also detect EEG signatures of post-stroke complications, such as seizures or cognitive impairments, enabling timely management and treatment adjustments.

### 3. Traumatic Brain Injury (TBI):

High-resolution EEG mapping can elucidate the neural correlates of cognitive, sensory, and motor impairments associated with TBI, guiding targeted rehabilitation strategies.

The system can monitor the progression of TBI-related EEG abnormalities over time and identify potential biomarkers of recovery or treatment response.

It can also inform the selection and optimization of neuromodulation interventions, such as transcranial direct current stimulation (tDCS) or neurofeedback, for enhancing cognitive and functional outcomes in TBI patients.

### 4. Neurodegenerative Diseases:

The PHAR system's ability to detect subtle changes in brain activity patterns can aid in the early diagnosis and monitoring of neurodegenerative disorders, such as Alzheimer's disease and Parkinson's disease.

It can identify EEG biomarkers associated with disease progression, cognitive decline, and motor symptoms, facilitating personalized disease management and treatment planning.

The system can guide the selection and titration of pharmacological treatments based on individual EEG profiles and monitor the effectiveness of disease-modifying therapies over time.

### 5. Psychiatric Disorders:

The PHAR system can identify EEG biomarkers associated with various psychiatric disorders, such as depression, anxiety, bipolar disorder, and schizophrenia.

It can help in the differential diagnosis of these conditions by detecting specific EEG patterns, such as frontal alpha asymmetry in depression or abnormal gamma oscillations in schizophrenia.

The system can guide the selection and titration of pharmacological treatments based on individual EEG profiles and monitor treatment response over time.

It can support combined pharmacological and neuromodulation therapies.

In also inform the targeting and optimization of neuromodulation interventions, such as transcranial magnetic stimulation (TMS) or transcranial direct current stimulation (tDCS), for treating psychiatric disorders.

### 6. Consciousness Disorders:

The PHAR system can assess the level of consciousness in patients with disorders of consciousness, such as coma, vegetative state, or minimally conscious state.

It can detect EEG signatures of residual cognitive function, such as event-related potentials or specific frequency band patterns, helping to differentiate between different states of consciousness.

The system can monitor the progression of consciousness disorders over time and identify potential signs of recovery or responsiveness to therapeutic interventions.

It can also guide the selection of appropriate brain-computer interface (BCI) technologies for communication and rehabilitation in patients with severe motor disabilities.

### 7. Brain Tumor Disorders:

The PHAR system can aid in the presurgical evaluation and surgical planning for patients with brain tumors.

It can map the functional cortex surrounding the tumor, identifying eloquent areas involved in language, motor, or sensory processing that should be preserved during surgery.

The system can detect EEG signatures of tumor-related epilepsy, guiding the resection of epileptogenic tissue and minimizing the risk of postoperative seizures.

It can also monitor the EEG activity during awake craniotomy procedures, providing real-time feedback to the neurosurgeon about the functional impact of tumor resection.

Postoperatively, the PHAR system can track the EEG changes associated with tumor recurrence or treatment-related side effects, informing timely interventions and follow-up care.

By leveraging the advanced EEG analysis capabilities of the PHAR system, clinicians can gain unprecedented insights into the neural mechanisms underlying a wide spectrum of neurological disorders. This information can guide personalized treatment strategies, optimize surgical interventions, and improve patient outcomes across various domains of neurological care. The system's ability to integrate with other IoMT devices and adapt to individual brain dynamics further enhances its potential to revolutionize the diagnosis and management of complex brain disorders.

### 5.2. Neuroscience Research and Brain-Computer Interfaces

The AI-powered EEG system with PHAR offers a powerful tool for neuroscience research, enabling the exploration of brain function and neural dynamics with unprecedented spatial and temporal resolution. Furthermore, its integration with automated reporting and adaptive neuromodulation capabilities paves the way for novel applications in brain-computer interfaces (BCIs) as noted in section 8020 of table 8000 in FIG. 8.

The AI-powered EEG system with PHAR has a wide range of applications in diagnosing, monitoring, and guiding treatment for various neurological disorders, as well as enhancing human performance and understanding brain function:

### 1. Epilepsy:

The PHAR system can precisely localize epileptogenic foci and map the spatiotemporal dynamics of seizure propagation, facilitating surgical planning and continuous monitoring of epileptic activity.

It can guide the placement of intracranial electrodes and the extent of surgical resection, improving the accuracy and efficacy of epilepsy surgery.

The system can also monitor the EEG activity during awake craniotomy procedures, providing real-time feedback to the neurosurgeon about the functional impact of surgical manipulations.

### 2. Stroke:

By accurately identifying the affected brain regions and monitoring the evolution of neural activity patterns, the system can assist in stroke diagnosis, prognosis, and tracking of recovery processes.

It can guide the selection of appropriate neurorehabilitation strategies based on the individual's EEG profiles and monitor the effectiveness of interventions over time.

The PHAR system can also detect EEG signatures of post-stroke complications, such as seizures or cognitive impairments, enabling timely management and treatment adjustments.

### 3. Traumatic Brain Injury (TBI):

High-resolution EEG mapping can elucidate the neural correlates of cognitive, sensory, and motor impairments associated with TBI, guiding targeted rehabilitation strategies.

The system can monitor the progression of TBI-related EEG abnormalities over time and identify potential biomarkers of recovery or treatment response.

It can also inform the selection and optimization of neuromodulation interventions, such as transcranial direct current stimulation (tDCS) or neurofeedback, for enhancing cognitive and functional outcomes in TBI patients.

### 4. Neurodegenerative Diseases:

The PHAR system's ability to detect subtle changes in brain activity patterns can aid in the early diagnosis and monitoring of neurodegenerative disorders, such as Alzheimer's disease and Parkinson's disease.

It can identify EEG biomarkers associated with disease progression, cognitive decline, and motor symptoms, facilitating personalized disease management and treatment planning.

The system can guide the selection and titration of pharmacological treatments based on individual EEG profiles and monitor the effectiveness of disease-modifying therapies over time.

### 5. Psychiatric Disorders:

The PHAR system can identify EEG biomarkers associated with various psychiatric disorders, such as depression, anxiety, bipolar disorder, and schizophrenia.

It can help in the differential diagnosis of these conditions by detecting specific EEG patterns, such as frontal alpha asymmetry in depression or abnormal gamma oscillations in schizophrenia.

The system can guide the selection and titration of pharmacological treatments based on individual EEG profiles and monitor treatment response over time.

It can support combined pharmacological and neuromodulation therapies.

It can also inform the targeting and optimization of neuromodulation interventions, such as transcranial magnetic stimulation (TMS) or transcranial direct current stimulation (tDCS), for treating psychiatric disorders.

### 6. Consciousness Disorders:

The PHAR system can assess the level of consciousness in patients with disorders of consciousness, such as coma, vegetative state, or minimally conscious state.

It can detect EEG signatures of residual cognitive function, such as event-related potentials or specific frequency band patterns, helping to differentiate between different states of consciousness.

The system can monitor the progression of consciousness disorders over time and identify potential signs of recovery or responsiveness to therapeutic interventions.

It can also guide the selection of appropriate brain-computer interface (BCI) technologies for communication and rehabilitation in patients with severe motor disabilities.

### 7. Brain Tumor Disorders:

The PHAR system can aid in the presurgical evaluation and surgical planning for patients with brain tumors.

It can map the functional cortex surrounding the tumor, identifying eloquent areas involved in language, motor, or sensory processing that should be preserved during surgery.

The system can detect EEG signatures of tumor-related epilepsy, guiding the resection of epileptogenic tissue and minimizing the risk of postoperative seizures.

It can also monitor the EEG activity during awake craniotomy procedures, providing real-time feedback to the neurosurgeon about the functional impact of tumor resection.

Postoperatively, the PHAR system can track the EEG changes associated with tumor recurrence or treatment-related side effects, informing timely interventions and follow-up care.

### 8. Cognitive Neuroscience:

Researchers can leverage the high-resolution EEG mapping to study the neural correlates of cognitive processes, such as attention, memory, decision-making, and language processing, with enhanced precision and reliability.

The PHAR system can provide insights into the spatiotemporal dynamics of cognitive functions, advancing our understanding of the brain-behavior relationships.

It can also facilitate the investigation of cognitive development, aging, and plasticity, by tracking EEG patterns across different age groups and in response to interventions.

### 9. Sensory and Motor Neuroscience:

The PHAR system can elucidate the intricate neural patterns underlying sensory perception, motor planning, and execution, advancing our understanding of these fundamental brain functions.

It can map the cortical representations of different sensory modalities, such as vision, audition, and somatosensation, with high spatial and temporal resolution.

The system can also investigate the neural mechanisms of motor learning, adaptation, and control, by monitoring EEG activity during various motor tasks and training paradigms.

### 10. Brain-Computer Interfaces (BCIs):

By decoding neural activity patterns in real-time, the system can facilitate the development of BCIs for applications ranging from assistive technologies to gaming and entertainment.

The PHAR system can enhance the accuracy and reliability of BCI control by providing high-resolution EEG features and adapting to individual brain dynamics.

It can also enable the design of more intuitive and user-friendly BCI interfaces by incorporating real-time feedback and adaptive algorithms.

### 11. Athletic Performance Enhancement:

The PHAR system can be used to monitor and optimize brain activity patterns associated with peak athletic performance, such as focus, decision-making, and motor control.

By providing real-time feedback and neurofeedback training, the system can help athletes develop mental skills and enhance their performance in various sports and physical activities.

It can also be used to assess and track the neural correlates of skill acquisition, expertise development, and performance under pressure, informing targeted training strategies.

### 12. Music Performance Enhancement:

The PHAR system can investigate the neural mechanisms underlying musical perception, creativity, and performance, by mapping the brain activity patterns associated with different aspects of musical experience.

It can provide insights into the effects of musical training on brain plasticity, cognitive functions, and emotional processing, informing educational and therapeutic interventions.

The system can also be used to develop neurofeedback protocols for enhancing musical skills, such as pitch perception, rhythm synchronization, and emotional expression.

### 13. Team Bonding and Collaboration:

By employing hyperscanning techniques, the PHAR system can simultaneously record and analyze the brain activity of multiple individuals during social interactions and collaborative tasks.

It can investigate the neural synchronization and coupling patterns associated with effective communication, coordination, and empathy within teams or dyads.

The system can provide insights into the factors that facilitate or hinder team bonding and collaboration, informing strategies for enhancing group performance and conflict resolution.

### 14. Neurofeedback Training:

The PHAR system can be used to deliver personalized neurofeedback training protocols, by providing real-time feedback of an individual's brain activity patterns.

It can target specific EEG features or brain networks associated with desired cognitive, emotional, or behavioral outcomes, such as attentional control, emotional regulation, or creativity.

The system can adapt the neurofeedback parameters based on the individual's progress and responses, optimizing the training effectiveness and efficiency.

Neurofeedback training with the PHAR system can be applied in various domains, including cognitive enhancement, stress management, and rehabilitation of neurological or psychiatric conditions.

By leveraging the advanced EEG analysis capabilities of the PHAR system, researchers and practitioners can gain unprecedented insights into the neural mechanisms underlying a wide range of human experiences and behaviors. This information can guide personalized interventions, optimize performance enhancement strategies, and advance our understanding of the brain's complex dynamics. The system's ability to integrate with other IoMT devices and adapt to individual brain patterns further enhances its potential to revolutionize the fields of neuroscience, psychology, and human performance optimization.

### 5.3. Personalized Medicine and Precision Neurology

The AI-powered EEG system with PHAR enables personalized medicine and precision neurology by tailoring diagnostics and treatments to individual brain anatomy and neurophysiology as noted in section 8030 of table 8000 in FIG. 8. The system's adaptability and integration with IoMT networks allow for the development of personalized treatment protocols and real-time monitoring of therapeutic responses.

Personalized Diagnostics: The PHAR system can adapt its referencing scheme and signal processing techniques to account for individual variations in brain anatomy and neural dynamics, ensuring accurate and personalized diagnosis.

Precision Neurology: By integrating real-time EEG analysis with adaptive neuromodulation capabilities, the system can adjust stimulation parameters based on individual responses, enabling precise and targeted interventions for various neurological conditions.

### 5.4. Remote Monitoring and Tele-Neurology

The integration of the AI-powered EEG system with IoMT networks facilitates remote monitoring and tele-neurology applications as noted in section 8040 of table 8000 in FIG. 8, expanding access to specialized neurological care and enabling continuous monitoring of patients in remote or underserved areas.

Remote Patient Monitoring: Patients can be monitored remotely, with real-time EEG data transmitted to healthcare providers for analysis and decision-making, reducing the need for frequent in-person visits.

Tele-Neurology Consultations: The automated EEG reporting capabilities enable efficient sharing of diagnostic information, allowing neurologists to provide remote consultations and expert opinions based on comprehensive EEG reports.

Continuous Monitoring and Early Intervention: The system's ability to detect and respond to changes in brain activity patterns facilitates continuous monitoring and early intervention, potentially preventing or mitigating the progression of neurological conditions.

The versatility of the AI-powered EEG system with PHAR, coupled with its integration with advanced technologies like AI, LLMs, and IoMT networks, unlocks a wide range of applications across clinical diagnostics, neuroscience research, personalized medicine, and remote healthcare delivery, ultimately contributing to improved patient outcomes and advancing our understanding of the human brain.

### 6. Future Directions and Enhancements

### 6.1. Expansion to Multi-Modal Neuroimaging Integration

While the AI-powered EEG system with Pathway Hierarchical Adaptive Referencing (PHAR) has demonstrated remarkable capabilities in high-resolution mapping of brain activity, future developments could involve the integration of multi-modal neuroimaging techniques. By combining EEG data with complementary modalities such as functional magnetic resonance imaging (fMRI), magnetoencephalography (MEG), and diffusion tensor imaging (DTI), the system could provide a more comprehensive understanding of brain function and neural dynamics.

Multi-modal integration would leverage the strengths of each neuroimaging technique, enabling the simultaneous assessment of both temporal and spatial aspects of brain activity. The high temporal resolution of EEG could be combined with the superior spatial resolution of fMRI and MEG, providing a more complete picture of the underlying neural processes. Additionally, DTI data could offer insights into the structural connectivity of the brain, further enhancing the system's ability to localize and characterize neural networks.

The integration of multi-modal neuroimaging data would require advanced signal processing and data fusion techniques, as well as sophisticated machine learning models capable of handling high-dimensional, multi-modal data. However, the potential benefits of this approach are significant, including improved diagnostic accuracy, enhanced understanding of brain-behavior relationships, and the development of more effective targeted interventions.

### 6.2. Incorporation of Explainable AI for Interpretability

While the AI-powered EEG system with PHAR leverages advanced machine learning models for tasks such as artifact removal, source localization, and pattern classification, there is a growing need for explainable AI (XAI) techniques to enhance the interpretability of the system's outputs. Explainable AI aims to provide transparency and insights into the decision-making process of machine learning models, enabling users to understand the rationale behind the system's predictions and recommendations.

The incorporation of XAI techniques would be particularly valuable in clinical settings, when interpretability and trust in the system's outputs are crucial. Explainable AI could provide clinicians with a better understanding of the neural patterns and features that are driving the system's diagnosis or treatment recommendations, enabling more informed decision-making and fostering confidence in the system's capabilities.

XAI techniques such as local interpretable model-agnostic explanations (LIME), SHapley Additive explanations (SHAP), and counterfactual explanations could be employed to elucidate the contributions of different EEG features and patterns to the system's outputs. Additionally, the integration of natural language processing (NLP) and visualization techniques could further enhance the interpretability of the system's results, enabling clinicians and researchers to gain deeper insights into the underlying neural processes.

### 6.3. Development of Standardized IoMT Protocols for Neuromodulation

The integration of the AI-powered EEG system with IoMT networks for adaptive neuromodulation represents a significant step towards personalized and precise interventions. However, to fully realize the potential of this approach, there is a need for the development of standardized protocols and guidelines for neuromodulation within the IoMT ecosystem.

Standardization efforts should focus on establishing common data formats, communication protocols, and security measures to ensure seamless interoperability between the EEG system, neuromodulation devices, and other IoMT components. This would facilitate the exchange of data and control signals, enabling real-time monitoring and adjustment of stimulation parameters across various devices and platforms.

Moreover, the development of standardized protocols for neuromodulation would promote consistency in treatment approaches, allowing for the sharing of best practices and the establishment of evidence-based guidelines. These protocols could encompass guidelines for stimulation parameter selection, safety considerations, and monitoring procedures, ensuring the effective and safe application of neuromodulation therapies across different clinical settings.

Collaboration between medical device manufacturers, healthcare providers, regulatory bodies, and research institutions would be essential in driving the development and adoption of these standardized protocols. By fostering a collaborative and standardized approach, the AI-powered EEG system with PHAR could more effectively integrate with the broader IoMT ecosystem, unlocking its full potential for personalized and adaptive neuromodulation.

### 6.4. Exploration of Novel Applications in Cognitive Enhancement and Wellness

While the primary focus of the AI-powered EEG system with PHAR has been on clinical diagnostics and therapeutic interventions, future developments could explore its potential applications in cognitive enhancement and overall wellness. As our understanding of the neural mechanisms underlying cognitive processes continues to evolve, the system's capabilities could be leveraged to modulate brain activity in ways that enhance cognitive functions such as attention, memory, and decision-making.

One potential application could be the development of personalized cognitive training programs, where the system's real-time EEG analysis and adaptive neuromodulation capabilities are used to tailor cognitive exercises to an individual's unique brain activity patterns. This approach could optimize the effectiveness of cognitive training by targeting specific neural networks and modulating brain activity in a precise and personalized manner.

Additionally, the AI-powered EEG system with PHAR could be explored for applications in stress reduction, mindfulness, and overall mental well-being. By monitoring brain activity patterns associated with stress, anxiety, and emotional regulation, the system could provide real-time feedback and adaptive neuromodulation interventions to promote relaxation and emotional balance.

Furthermore, the integration of the system with virtual reality (VR) and augmented reality (AR) technologies could open up new avenues for immersive cognitive enhancement and wellness experiences. By combining real-time EEG analysis with adaptive neuromodulation and immersive environments, users could potentially engage in guided meditation, cognitive training, or stress-reduction exercises tailored to their individual brain activity patterns.

To explore these novel applications, interdisciplinary collaborations between neuroscientists, psychologists, cognitive scientists, and technology developers would be essential. Additionally, ethical considerations and regulatory frameworks would need to be established to ensure the responsible and safe use of the AI-powered EEG system with PHAR for cognitive enhancement and wellness purposes.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

We claim:

1. An AI-powered EEG system comprising:
an ultra-dense electrode array with 16 to 1024 EEG sensors;
a pathway hierarchical adaptive referencing circuit for localizing EEG detection, including:
a) a multi-layered multiplexer hierarchy accommodating EEG sensor inputs and dynamically configuring sensor groupings,
b) parallel processing units for evaluating optimal referencing topologies across hierarchical layers, and
c) control logic for adaptive configuration adjustments based on real-time EEG signal characteristics;
an AI component for EEG noise reduction, artifact removal, source localization and classification; and
a central processing unit and memory for executing AI algorithms and controlling system operations.

2. The system of claim 1, further comprising one or a plurality of elements from the group consisting of a flexible pogo pin electrode array enabling adaptive spatial sampling and high-density EEG acquisition, a transcranial electrical stimulation (tES) component for delivering targeted neuromodulation, an electrical impedance tomography (EIT) component for impedance-based brain imaging, and a user interface for visualizing high-resolution EEG activity maps and generating reports.

3. The system of claim 1, wherein the AI component includes one or a plurality of networks selected from the group consisting of convolutional neural networks (CNNs) for extracting features from EEG topographies, recurrent neural networks (RNNs) for capturing temporal dependencies and sequential patterns in EEG time series, generative adversarial networks (GANs) for generating realistic EEG data for data augmentation and simulation, and graph neural networks (GNNs) for modeling and analyzing the complex graph-structured relationships between EEG channels, cortical regions, and functional brain networks.

4. The system of claim 1, further comprising a large language model (LLM) for automatically generating human-readable EEG reports, wherein the LLM is fine-tuned on a corpus of expert-annotated EEG reports and corresponding EEG data.

5. The system of claim 1, further comprising Internet of Medical Things (IoMT) connectivity for integrating with one or a plurality of external devices selected from the group consisting of: neuromodulation devices, wearable sensors, electronic health records (EHRs), and remote monitoring systems.

6. The system of claim 5, wherein the IoMT connectivity enables closed-loop adaptive neuromodulation, with real-time EEG analysis guiding the adjustment of stimulation parameters in connected neuromodulation devices.

7. The system of claim 1, wherein the ultra-dense electrode array has a sensor density of 128-1024 electrodes and an inter-electrode spacing of 5-20 mm or less.

8. The system of claim 1 where the AI-powered EEG component is utilized for analyzing EEG signals functioning by
acquiring high-density EEG data from the electrode array;
dynamically adjusting referencing configurations using the pathway hierarchical adaptive referencing circuit;
applying AI machine learning models for EEG noise reduction, artifact removal, source localization and classification;
generating and visualizing real-time, high-resolution EEG activity maps; and
classifying EEG spatiotemporal patterns to decode neural dynamics and brain states.

9. The system of claim 8, further comprising automatically generating human-readable EEG reports using the integrated large language model (LLM).

10. The system of claim 8, further comprising providing closed-loop adaptive neuromodulation by:
analyzing real-time EEG data to infer the current brain state and treatment response;
determining optimal stimulation parameters based on the EEG analysis; and
adjusting stimulation parameters of connected IoMT neuromodulation devices according to the determined optimal parameters.

11. The system of claim 1 where the functions are implemented in hardware consisting of a multiplicity of components selected from the group consisting of Power Module, Central Processing Unit, RAM storage, ROM storage, Mass-Storage Subsystem, parallel processors, Artificial Intelligence Processor, Encryption Processor, User-Interface Controller, External Communications Processor, Analog Front End, EEG Input Array, Electrical Stimulation Output Controller, and Stimulation Output Array in which communications between components are handled by one or a plurality of mechanisms selected from the group consisting of Communications Bus and direct and the components and where electrodes for both EEG input and stimulation output can be shared, typically by use of a multiplexer.
